(19) United States
(12) Patent Application Publication
(10) Pub. No.: US 2025/0032340 A1
(43) Pub. Date: Jan. 30, 2025

(54) METHOD, APPARATUS, AND PROGRAM FOR HIGH-SPEED OPTIMIZATION OF AUXILIARY PROFILE OF MOBILE WEARABLE ROBOT

(71) Applicant: HUROTICS Inc., Seoul (KR)

(72) Inventors: Seungtae YANG, Suwon-si (KR); Kimoon NAM, Gunpo-si (KR); Saikat SAHOO, Seoul (KR)

(73) Assignee: HUROTICS Inc., Seoul (KR)

(21) Appl. No.: 18/783,211

(22) Filed: Jul. 24, 2024

(30) Foreign Application Priority Data
Jul. 25, 2023 (KR) 10-2023-0096578
Mar. 7, 2024 (KR) 10-2024-0032574
Mar. 7, 2024 (KR) 10-2024-0032575
Mar. 7, 2024 (KR) 10-2024-0032576

(51) Int. Cl. A61H 3/00; A61H 1/02; B25J 9/00; G16H 40/67

(52) U.S. Cl. CPC A61H 3/00; A61H 1/0244; B25J 9/0006; G16H 40/67; A61H 2201/5007; A61H 2230/625

(57) ABSTRACT

Provided is a method of high-speech optimization of an auxiliary profile of a mobile wearable robot. The method includes providing assistance power to a user to help the user's walk, collecting body response information of the user measured after the providing of the assistance power, performing high-speed optimization on an auxiliary profile corresponding to the assistance power based on the body response information, and providing the user with optimal assistance power corresponding to the optimal auxiliary profile on which the high-speed optimization has been performed.

### CROSS-REFERENCE TO RELATED APPLICATION

This application claims priority to and the benefit of Korean Patent Application Nos. 10-2023-0096578, filed on Jul. 25, 2023, 10-2024-0032574, filed on Mar. 7, 2024, 10-2024-0032576 filed on Mar. 7, 2024, and 10-2024-0032575, filed on Mar. 7, 2024, the disclosure of which is incorporated herein by reference in its entirety.

### BACKGROUND

### 1. Field of the Invention

The present invention relates to a method, an apparatus, and a program for high-speed optimization of an auxiliary profile of a mobile wearable robot, and specifically, to a method, an apparatus, and a program for performing high-speed optimization on an auxiliary profile based on body response information collected in real time.

### 2. Discussion of Related Art

A wearable robot is a device that is worn on a user as clothing and assists the user's movement by transmitting assistance power from a driving part, such as a motor, to the clothing. Such a wearable robot assists a user who needs walking assistance and rehabilitation treatment to easily carry out daily lives or receive treatment, and when compared to the existing exoskeleton-type rehabilitation robots, has a low weight and volume, thereby making it easy to handle and wear.

However, the conventional wearable robots, when applying assistance power to a user, provide uniform assistance power rather than adjusting the assistance power while examining the effect of the assistance, that is, it provides assistance power in a one-sided form. Since the conventional wearable robots provide assistance power in a one-sided form, which results in a decrease in the effectiveness of the assistance and also a decrease in the effectiveness in daily life or during treatment.

Therefore, there is a need to develop an algorithm capable of, while examining and monitoring the actual effects of providing assistance power to a user wearing a wearable robot, adjusting a magnitude, timing, and pattern of the assistance power according to a body response of the user. As the related arts, there is disclosed Korean Patent Registration No. 10-2416734 titled “wearable assistance apparatus.”

### SUMMARY OF THE INVENTION

The present invention is directed to providing a method, an apparatus, and a program for high-speed optimization of an auxiliary profile of a mobile wearable robot.

The technical objectives of the present invention are not limited to the above, and other objectives that are not described above may become apparent to those of ordinary skill in the art based on the following descriptions.

According to an aspect of the present invention, there is provided a method of high-speech optimization of an auxiliary profile of a mobile wearable robot. The method includes providing assistance power to a user to help the user's walk, collecting body response information of the user measured after the providing of the assistance power, performing high-speed optimization on an auxiliary profile corresponding to the assistance power based on the body response information, and providing the user with optimal assistance power corresponding to the optimal auxiliary profile on which the high-speed optimization has been performed.

The collecting of the body response information of the user in real time may include collecting the body response information including an angular velocity measured in a sagittal plane of a thigh of the user, a delivered force corresponding to the assistance power, and a force timing related to a time for delivering the assistance power to the user.

The angular velocity may include an angular velocity for the sagittal plane of the thigh of the user measured by an inertial measurement device attached to the thigh of the user, the delivered force may include a force exerted by a mobile wearable robot worn on the user to pull the thigh of the user in response to the assistance power, and the force timing may include a time of providing the assistance power to the user based on a gait cycle percentage recognized from a difference between a first time at which a maximum angle is recognized by the inertial measurement device and a second time at which a next maximum angle is recognized.

The performing of the high-speed optimization on the auxiliary profile corresponding to the assistance power based on the body response information may include calculating augmentation power based on the angular velocity, the delivered force, and the force timing, deriving an optimal force timing at which the augmentation power is maximized, and generating the optimal auxiliary profile by reflecting the optimal force timing in the auxiliary profile.

The calculating of the augmentation power based on the angular velocity, the delivered force, and the force timing may include recognizing a delivered force transmitted to the user correspondingly to the force timing during the gait cycle percentage of the user, recognizing a moment arm, which is a distance between a point of action of a body of the user corresponding to the delivered force and a center of gravity of the thigh of the user, performing integration on a value obtained by multiplying the delivered force and the moment arm and recognizing augmentation work generated during the gait cycle percentage by the delivered force, and calculating the augmentation power by dividing the augmentation work by a time corresponding to the gait cycle percentage.

The deriving of the optimal force timing at which the augmentation power is maximized may include obtaining a dataset including a plurality of force timings previously provided to the user and augmentation power corresponding to the plurality of force timings, respectively, and repeatedly performing a Bayesian optimization technique based on the dataset to derive a force timing that maximizes an expected augmentation power value.

The generating of the optimal auxiliary profile by reflecting the optimal force timing in the auxiliary profile may include adjusting parameters of the auxiliary profile based on the optimal force timing, and the parameters of the auxiliary profile may include a point in time at which a delivered force is generated, a point in time at which a magnitude of the delivered force is greatest, and a point in time at which the delivered force becomes 0 again.

The method may further include, after the providing of the optimal assistance power to the user, performing high-speed optimization again using body response information of the user measured after the providing of the optimal assistance power, and providing the user with assistance power corresponding to the optimal assisting profile on which the high-speed optimization has been performed, wherein the optimal auxiliary profile may be optimized in real time and updated.

The method may further include obtaining sensing data according to walking of the user from an inertial measurement device attached to the thigh of the user, defining a plurality of gait feature points based on the sensing data, determining a specific gait feature point corresponding to a current walking environment of the user based on the plurality of gait feature points and reference values preset for a plurality of walking environments, respectively, and predicting a gait cycle percentage of the user based on the specific gait feature point.

The method may further include generating a first function corresponding to a metabolic energy and a second function corresponding to a robotic energy, recognizing a Pareto front that minimizes each of the metabolic energy and the robot energy based on the first function and the second function, adding a smooth value to an auxiliary profile based on the Pareto front to obtain an optimal auxiliary profile, and providing the user with assistance power corresponding to the optimal auxiliary profile.

According to an aspect of the present invention, there is provided an apparatus including a memory in which one or more instructions are stored, and a processor configured to execute the one or more instructions stored in the memory, wherein the processor performs the method according to claim 1 by executing the one or more instructions.

According to an aspect of the present invention, there is provided a computer-readable recording medium. The computer-readable recording medium may provide a surgery simulation method in combination with a computer which is hardware.

Other specific details according to the present invention are included in the specification and the accompanying drawings.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a diagram illustrating a system according to an embodiment of the present invention;

FIG. 2 is a hardware configuration diagram of a computing device according to an embodiment of the present invention;

FIGS. 3 to 8 are diagrams for describing an example of a method of high-speed optimization of an auxiliary profile of a mobile wearable robot according to an embodiment of the present invention;

FIG. 9 is a diagram illustrating an example of a method of predicting a gait cycle percentage corresponding to various walking environments according to various embodiments according to the present invention; and

FIG. 10 is a diagram illustrating an example of a method of simultaneously optimizing the energy efficiency of a wearable robot and the metabolic energy efficiency of a wearer according to various embodiments according to the present invention.

### DETAILED DESCRIPTION OF EXEMPLARY EMBODIMENTS

Various embodiments are described with reference to the accompanying drawings. In this specification, various descriptions are presented to provide an understanding of the present invention. However, it may be apparent that the embodiments may be practiced without detailed descriptions thereof.

As used herein, the terms “component,” “module,” “system,” etc., refer to a computer-related entity, hardware, firmware, software, a combination of software and hardware, or an implementation of software. For example, a component may be, but is not limited to, a process running on a processor, a processor, an object, a thread of execution, a program, and/or a computer. For example, both an application running on a computing device and the computing device can each be a component. One or more components may reside within a processor and/or thread of execution. A component may be localized within one computer. A component may be distributed between two or more computers. Additionally, these components can execute from various computer-readable media having various data structures stored thereon. Components can communicate through local and/or remote processing depending on signals, for example, with one or more data packets (e.g., data transmitted through data and/or signals from one component interacting with other components in a local system, a distributed system, to other systems and over a network such as the Internet).

In addition, the term “or” is intended to mean an inclusive “or” and not an exclusive “or.” That is, unless otherwise specified or clear from context, “X uses A or B” is intended to mean one of the natural implicit substitutions. That is, either X uses A; X uses B; or, when X uses both A and B, “X uses A or B” can apply to either of these cases. Additionally, the term “and/or” used herein should be understood to refer to and include all possible combinations of one or more of the related items listed.

The terms “comprise,” “comprising,” “include,” and/or “including” used herein specify the presence of stated features, integers, steps, operations, elements, components and/or groups thereof and do not preclude the presence or addition of one or more other features, integers, steps, operations, elements, components, and/or groups thereof. It should be understood that the singular forms “a” and “an” in addition include the plural forms unless the context clearly dictates otherwise.

Those skilled in the art will additionally recognize that the various illustrative logical blocks, configurations, modules, circuits, means, logic, and algorithm steps described in connection with the embodiments disclosed herein may be implemented in electronic hardware, computer software, or both. It should be recognized that combinations can be implemented. To clearly illustrate the interchangeability of hardware and software, various illustrative components, blocks, configurations, means, logics, modules, circuits, and steps have been described above generally in terms of their functionality. Whether such functionality is implemented in hardware or software will depend on the specific application and design constraints imposed on the overall system. A skilled technician can implement the described functionality in a variety of ways for each specific application. However, such implementation decisions should not be construed as causing a departure from the scope of the present disclosure.

The description of the presented embodiments is provided to enable those skilled in the art to use or practice the present disclosure. Various modifications to these embodiments will be apparent to those skilled in the art. The general principles defined herein may be applied to other embodiments without departing from the scope of the disclosure. Therefore, the present disclosure is not limited to the embodiments presented herein. This disclosure is to be interpreted in the broadest scope consistent with the principles and novel features presented herein.

In the specification, a computer may be any type of hardware device including at least one processor, and may be understood to encompass software components operating in the corresponding hardware device according to embodiments. For example, a computer may be understood to be a smart phone, a tablet PC, a desktop, a notebook, a user client, or an application running on any of these devices, but is not limited thereto.

Hereinafter, embodiments of the present invention will be described in detail with reference to the accompanying drawings.

Each operation described in the specification is given as being performed by a specific component or computer, but the agent of each operation is not limited thereto, and at least some of the operations may be performed in different devices according to embodiments.

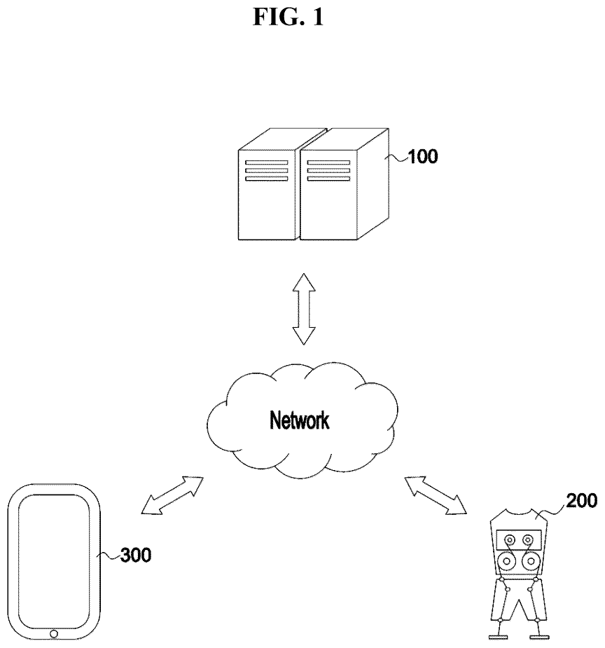

FIG. 1 is a diagram illustrating a system according to an embodiment of the present invention.

Referring to FIG. 1, a system according to the embodiment of the present invention may include a computing device 100, a wearable robot 200, and a user terminal 300. The system shown in FIG. 1 is provided according to one embodiment, and components thereof are not limited to the embodiment shown in FIG. 1, and may be added, changed, or deleted when required.

The computing device 100 according to the present invention may control the wearable robot 200. Additionally, the wearable robot 200 may be controlled through the user terminal 300 carried by a user. In addition, the user terminal 300 may receive various types of information related to adjustment of the wearable robot 200 through the computing device 100.

According to an embodiment of the present invention, due to variations in body shapes and walking patterns among users, the walking assistance effects for the same auxiliary profile may differ for each user. Additionally, the optimal auxiliary profile that maximizes the walking assistance effect may vary depending on users. Therefore, in order to obtain the optimal auxiliary profile, there is a need for optimization of an auxiliary profile for each user.

Accordingly, the present invention discloses a method, an apparatus, and a program for optimizing an auxiliary profile for each user at high speed.

In one embodiment, the computing device 100 may perform high-speed optimization on an auxiliary profile of a mobile wearable robot.

Specifically, the computing device 100 may provide a user with assistance power to help the user's walk. Additionally, the computing device 100 may collect body response information of the user that is measured after providing the assistance power. Additionally, the computing device 100 may perform high-speed optimization on an auxiliary profile corresponding to the assistance power based on the body response information. Additionally, the computing device 100 may provide the user with optimal assistance power corresponding to the optimal auxiliary profile on which the high-speed optimization has been performed.

Accordingly, the computing device 100 may provide each user with an optimized walking experience through personalized walking assistance. This may improve the user's walking stability and efficiency, and by providing customized assistance to users with various body shapes and walking patterns, expand the scope of application of walking assistance technology and improve user satisfaction.

In various embodiments, the computing device 100 may provide a user with a specific assistance power to help the user's walk. Specifically, the computing device 100 may control the wearable robot 200 to provide the user with the specific assistance power.

The computing device 100 may collect body response information of the user. Specifically, the computing device 100 may collect information about the cost of the user using the metabolic energy when the specific assistance power is provided. More specifically, the computing device 100 may collect the cost of the user using a breath-based metabolic energy.

The computing device 100 may perform high-speed optimization on an auxiliary profile based on the body response information collected in real time. Specifically, the computing device 100 may perform Bayesian optimization based on a power-based indicator related to an assistance performance of the wearable robot.

The computing device 100 may provide the user with assistance power corresponding to the auxiliary profile on which the high-speed optimization has been performed. Specifically, the computing device 100 may control the wearable robot 200 to provide assistance power corresponding to the auxiliary profile on which the high-speed optimization has been performed.

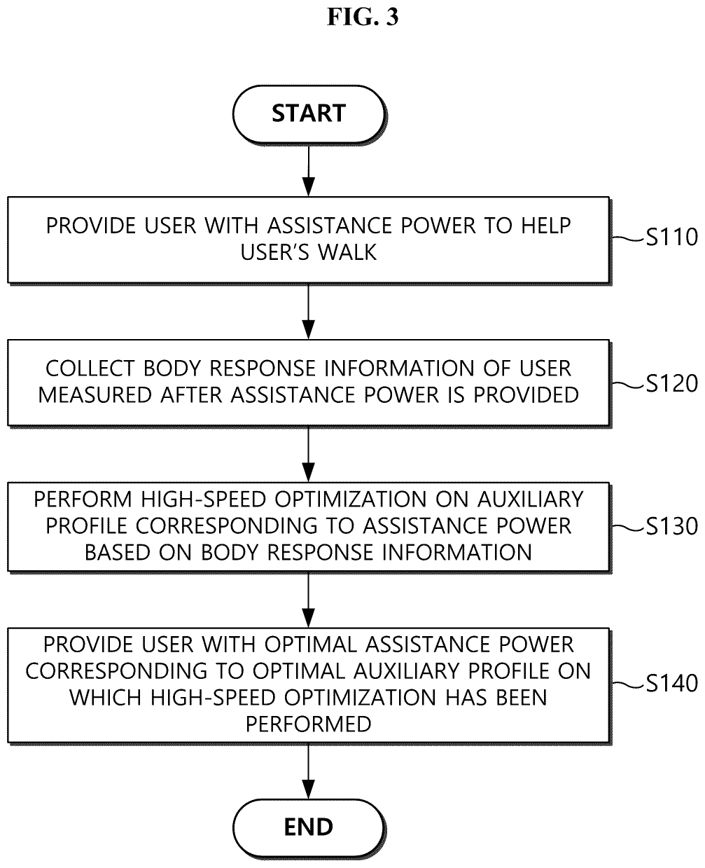

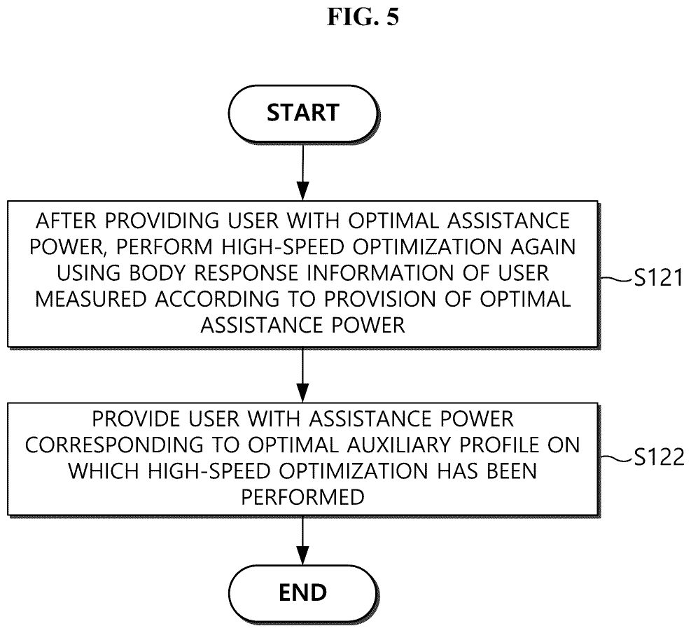

An example of a method of the computing device 100 performing high-speed optimization on an auxiliary profile of a mobile wearable robot will be described below with reference to FIGS. 3 to 8.

According to one embodiment of the present invention, it may be important for a wearable robot to transmit power at a precise timing to help the user's walk. To this end, knowing the accurate auxiliary profile is important but predicting the accurate gait cycle percentage may also be important.

Most of the existing, widely used gait cycle percentage prediction algorithms accurately operate for a single walking environment, and thus has a limitation that the accuracy is low in various other walking environments. In addition, there are a large number of existing gait cycle percentage prediction algorithms that use deep learning to predict an accurate gait cycle percentage, but the algorithms also have limitations in predicting various gait cycle percentages, and due to the heavy computational requirements of deep learning, it is difficult to load the algorithm in a mobile wearable robot.

Accordingly, the computing device 100 according to the present invention may accurately predict the gait cycle percentage in various walking environments, such as flat ground, uphill, and running, using a single sensor (for example, an inertial measurement sensor or an inertial measurement unit).

In one embodiment, the computing device 100 may predict the gait cycle percentage corresponding to various walking environments.

Specifically, the computing device 100 may obtain sensing data according to the user's walking from the inertial measurement device attached to the user's thigh. Additionally, the computing device 100 may define a plurality of gait feature points based on the sensing data. Additionally, the computing device 100 may determine a specific gait feature point corresponding to a current walking environment of the user based on the plurality of gait feature points and reference values preset for each of a plurality of walking environments. Additionally, the computing device 100 may predict the gait cycle percentage of the user based on the specific gait feature point.

In the present invention, the specific gait feature point may be a time point or position recognized as a heel strike. Here, the heel strike may represent the moment when the heel touches the ground during walking, and may be used to identify a start and an end of the gait cycle percentage.

The computing device 100 according to the present invention may accurately identify and analyze various gait feature points including the heel strike, thereby providing optimized support tailored to the user's walking pattern and the environment through the wearable robot 200.

In addition, the present invention may identify special requirements in various walking environments, such as walking on an uphill, walking on a flat ground, or running, through in-depth analysis related to the user's walking pattern, and respond to the special requirements. Through this, the wearable robot 200 may provide more effective assistance to the user, improving walking efficiency, and reducing the risk of potential walking-related injuries.

In addition, through an interface provided through the user terminal 300, the user may check important information, such as an operating status of the wearable robot 200, a battery level, walking data analysis results, etc., in real time. The information may allow the user to better understand the use of the wearable robot 200 and adjust the wearable robot 200 as needed, thereby contributing to improved user experience.

The present invention may play an important role in improving the quality of daily life of various users, including the elderly and patients in need of rehabilitation, by providing personalized walking assistance.

The computing device 100 according to the present invention may obtain sensing data according to the user's walking from a gait cycle percentage measurement sensor.

Specifically, the computing device 100 may receive sensing data measured by the gait cycle percentage measurement sensor provided in the wearable robot 200 from the wearable robot 200. Here, the gait cycle percentage measurement sensor may include an inertial measurement sensor for measuring a hip angle of the user.

The computing device 100 may recognize a current walking environment of the user based on the sensing data and reference values preset according to walking environments. Additionally, the computing device 100 may recognize gait feature points based on the current walking environment. Additionally, the computing device 100 may predict the gait cycle percentage based on the gait feature point.

Specifically, the computing device 100 may recognize signal characteristics of the gait cycle percentage measurement sensor to predict the gait cycle percentage. Additionally, the computing device 100 may predict the gait cycle percentage based on the signal characteristics. Additionally, the computing device 100 may calculate an error in the predicted gait cycle percentage. Additionally, the computing device 100 may correct the predicted gait cycle percentage by compensating for the error.

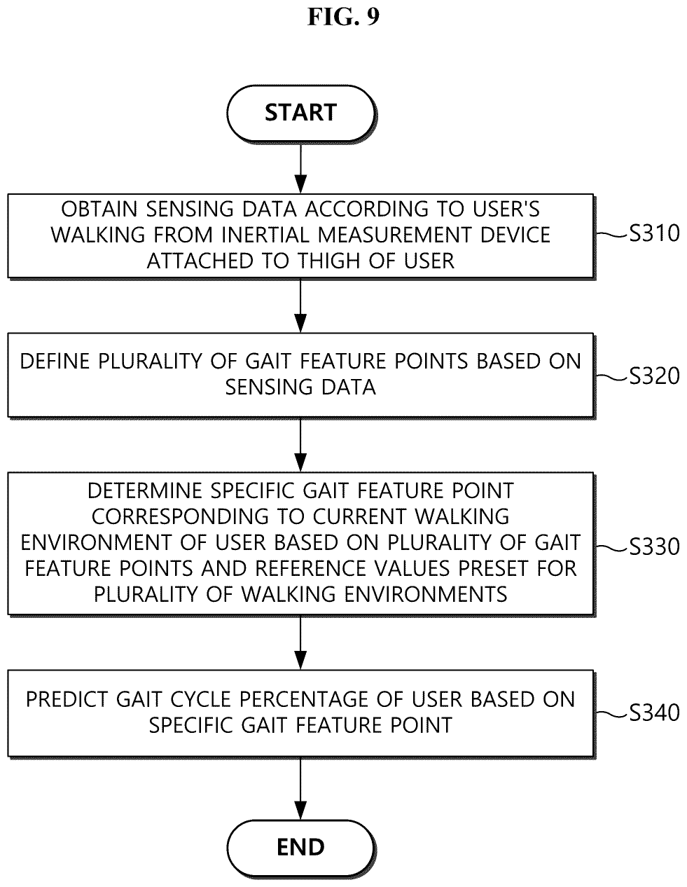

A method of the computing device 100 predicting a gait cycle percentage corresponding to various walking environments will be described below with reference to FIG. 9.

With an embodiment of the present invention, in the field of wearable robots for helping users walk, various issues may arise in the wearable robot when the wearable robot is driven by considering only the user (specifically, metabolic energy of the user). In particular, in the case of mobile wearable robots, when driven by considering only the user, battery shortage or heat generation may occur. For example, when a wearable robot provides a strong assistance power by considering only the user, there are issues such as reduced battery efficiency and motor efficiency, and furthermore, the operating time of the wearable robot is shortened.

Accordingly, the computing device 100 according to the present invention may simultaneously optimize the energy efficiency of the wearable robot and the metabolic energy efficiency of the wearer.

In one embodiment, the computing device 100 may generate a first function corresponding to metabolic energy and a second function corresponding to robotic energy. Additionally, the computing device 100 may recognize a Pareto front that minimizes each of the metabolic energy and the robotic energy based on the first function and the second function. Additionally, the computing device 100 may add a smooth value to an auxiliary profile based on the Pareto front, to obtain an optimal auxiliary profile. Additionally, the computing device 100 may provide the user with assistance power corresponding to the optimal auxiliary profile.

Accordingly, the computing device 100 may optimize the interaction between the user and the wearable robot 200 and provide customized feedback and control options to the user through the user terminal 300, thereby maximizing the usability of the wearable robot and improving energy efficiency. As a result, the wearable robot 200 may extend battery life and reduce heat generation while providing more effective assistance power by considering the user's metabolic energy.

In various embodiments, the computing device 100 according to the present invention may add a smooth value to an auxiliary profile including an Onset point, a Peak point, and a Release point to correct the auxiliary profile.

Specifically, the computing device 100 may add at least one of Offset X and Offset Y to each of the Onset point, the Peak point, and the Release point. Here, Offset X and Offset Y may each be determined based on a preset optimization algorithm.

The computing device 100 may, upon correction of the auxiliary profile, provide the user with assistance power corresponding to the corrected auxiliary profile.

Specifically, the computing device 100 may control the wearable robot 200 to provide assistance power corresponding to the corrected auxiliary profile.

In various embodiments, the computing device 100 according to the present invention may provide a plurality of assistance modes. In this case, the computing device 100 may consider the energy efficiency of the wearable robot and the metabolic energy efficiency of the wearer.

Specifically, the computing device 100 may perform multi-objective Bayesian optimization using a first objective function corresponding to the energy efficiency of the wearable robot and a second objective function corresponding to the metabolic energy efficiency of the wearer. Additionally, the computing device 100 may generate an auxiliary profile corresponding to at least one mode based on the optimized result. Here, the at least one mode may include at least one of a power mode that maximizes the wearer's metabolic energy efficiency, an eco-mode that maximizes the wearable robot's energy efficiency, and a smart mode that maximizes each of the wearable robot's energy efficiency and the wearer's metabolic energy efficiency, but is not limited thereto.

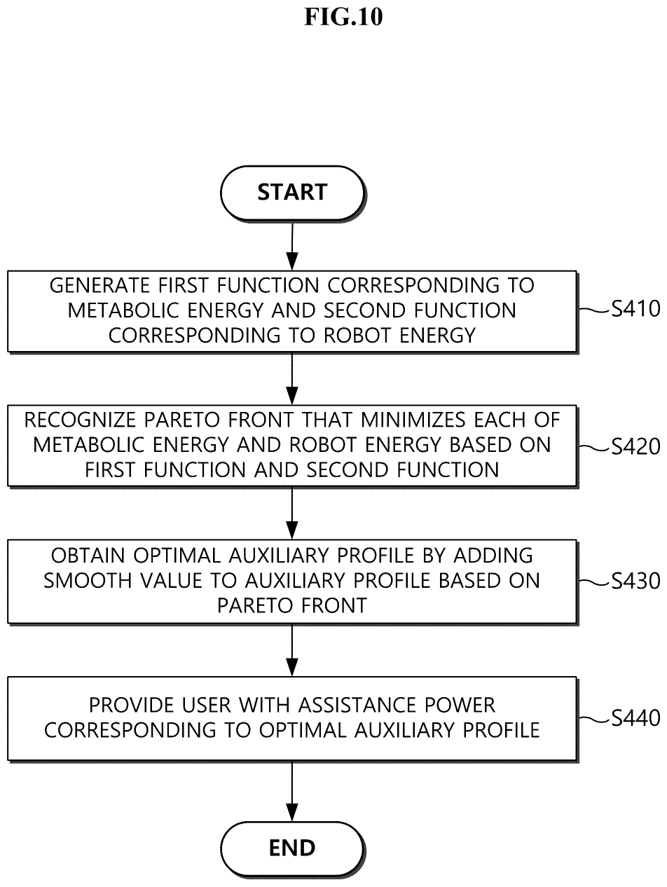

A method of the computing device 100 simultaneously optimizing the energy efficiency of the wearable robot and the metabolic energy efficiency of the wearer will be described below with reference to FIG. 10.

The computing device 100 is a data management device related to drive control of the wearable robot 200. To this end, the computing device 100 controls a driving unit that moves the wearable robot 200 based on sensing data and exercise data. In detail, the computing device 100 obtains physical ability information, determines the fitness level of the user based on the physical ability information, obtains information regarding an adjustment direction of the driving unit, and obtains driving signal information corresponding to the adjustment direction based on the determined fitness level of the user.

The wearable robot 200 is configured to assist the user with the power required for exercise such as walking while worn by the user. For this, the wearable robot 200 includes one or more members that may be worn on the user body and a driving unit that may be driven while connected to the member with a wire and apply power by winding and unwinding the wire. Additionally, the wearable robot 200 may be further provided with a sensor unit connected to the driving unit to obtain sensing data and exercise data.

The user terminal 300 is configured to receive various types of information that may be obtained during the power assistance process of the wearable robot 200 and input whether to enter the assistance mode after wearing the wearable robot 200, and is provided as a terminal carried by the user. To this end, the user terminal 300 may include, on at least a portion thereof, a touchable display in which various types of information may be visually checked and input, and various user interfaces (UIs) (for example, an assistance mode decision UI, a fitness level UI, control information UI, etc.) provided by the computing device 100 may be output through the display. For example, the user terminal 300 may include at least one of a smartphone, a tablet PC, a laptop computer, and a desktop computer, and may be provided with the above-described UI implemented on a web page or app page from the computing device 100, but is limited thereto.

In various embodiments, the system according to the present invention may include an external server (not shown). The external server may be connected to the computing device 100 through a network, and may transmit and receive various information/data required for the computing device 100 to control the wearable robot 200, and may store and manage various information/data generated as the computing device 100 controls the wearable robot 200.

For example, the external server may be a database server that stores information used to control the wearable robot 200. For another example, the external server may be a server that provides information used to control the wearable robot 200.

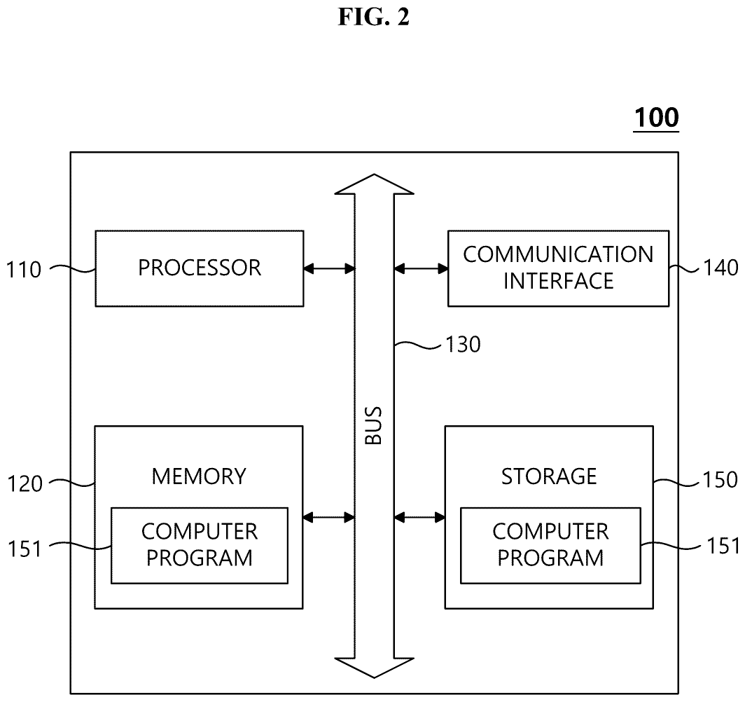

FIG. 2 is a hardware configuration diagram of a computing device according to an embodiment of the present invention.

Referring to FIG. 2, the computing device 100 according to an embodiment of the present invention may include one or more processors 110, a memory 120 that loads a computer program 151 executed by the processor 110, a bus 130, a communication interface 140, and a storage 150 that stores the computer program 151. Here, only components related to the embodiment of the present invention are shown in FIG. 2. Accordingly, it should be understood by those skilled in the art to which the present invention pertains that other general-purpose components may be included in addition to the components shown in FIG. 2.

The processor 110 controls the overall operation of each component of the computing device 100. The processor 110 may include one or more cores, and may include processors of the computing device, such as a central processing unit (CPU), a general purpose graphics processing unit (GPGPU), and a tensor processing unit (TPU) of the computing device and other processors for data analysis and deep learning. Alternatively, the processor 110 may include any type of processor well known in the art of the present invention.

Additionally, the processor 110 may perform operations on at least one application or program for executing methods according to embodiments of the present invention, and the computing device 100 may include one or more processors.

In various embodiments, the processor 110 may further include a random access memory (RAM) (not shown) and a read-only memory (ROM) (not shown) that temporarily and/or permanently store signals (or data) processed in the processor 110. Additionally, the processor 110 may be implemented in the form of a system on chip (SoC) that includes at least one of a GPU, a RAM, and a ROM.

The memory 120 stores various data, commands and/or information. The memory 120 may download the computer program 151 from the storage 150 to execute methods/operations according to various embodiments of the present invention. When the computer program 151 is downloaded into the memory 120, the processor 110 may perform the method/operation by executing one or more instructions constituting the computer program 151. The memory 120 may be implemented as a volatile memory, such as an RAM, but the technical scope according to the present invention is not limited thereto.

The bus 130 provides communication functionality between components of the computing device 100. The bus 130 may be implemented as various types of buses, such as an address bus, a data bus, and a control bus.

The communication interface 140 supports wired and wireless Internet communication of the computing device 100. Additionally, the communication interface 140 may support various communication methods other than Internet communication. To this end, the communication interface 140 may be configured to include a communication module well known in the technical field according to the present invention. In some embodiments, the communication interface 140 may be omitted.

The storage 150 may non-temporarily store the computer program 151. When performing a process according to an embodiment of the present invention through the computing device 100, the storage 150 may store various information required to perform analysis according to the disclosed embodiment.

The storage 150 may include a non-volatile memory, such as an ROM, an erasable programmable ROM (EPROM), an electrically erasable programmable ROM (EEPROM), and flash memory, a hard disk, a removable disk, or any other type of computer-readable recording medium well known in the art to which the present invention pertains.

The computer program 151 may include one or more instructions that, when loaded into the memory 120, cause the processor 110 to perform methods/operations according to various embodiments of the present invention. That is, the processor 110 may perform the method/operation according to various embodiments according to the present invention by executing the one or more instructions.

In one embodiment, the computer program 151 may include one or more instructions to perform various methods related to various tasks associated with training a neural network model.

The operations of a method or algorithm described in connection with an embodiment of the present invention may be implemented directly by hardware, by a software module executed by hardware, or by a combination of hardware and a software module. The software module may reside in an RAM, an ROM, an EPROM, an EEPROM, a flash memory, a hard disk, a removable disk, a compact disc read only memory (CD-ROM), or any other type of computer-readable recording medium well known in the art to which the present invention pertains.

The components of the present invention may be implemented as a program (or application) to be executed in combination with a computer, which is hardware, and may be stored in a medium. The components of the present invention may be implemented with software programming or software components, and similarly, embodiments may be implemented with programming or scripting languages, such as C, C++, Java, assembler, etc., including various algorithms implemented in a combination of data structures, processes, routines or other programming components. Functional aspects may be implemented with algorithms running on one or more processors.

FIGS. 3 to 8 are diagrams for describing an example of a method of high-speed optimization of an auxiliary profile of a mobile wearable robot according to an embodiment of the present invention.

Referring to FIG. 3, the computing device 100 may provide the user with assistance power to help the user's walk through control of the wearable robot 200 (S110).

Specifically, when there is body response information previously measured from the user, the computing device 100 may provide the user with assistance power corresponding to an optimized auxiliary profile based on the previously measured body response information. That is, operations S110 to S140 shown in FIG. 3 may be performed repeatedly.

Meanwhile, when there is no body response information previously measured from the user, the computing device 100 may provide the user with assistance power according to a randomly generated force timing.

The computing device 100 may collect body response information of the user measured after providing the assistance power (S120).

Specifically, the computing device 100 may, when collecting the body response information of the user in real time, collect body response information including an angular velocity measured in a sagittal plane of a thigh of the user, a delivered force corresponding to the assistance power, and an force timing related to a time of delivering the assistance power to the user.

In one embodiment, the angular velocity may include an angular velocity for the sagittal plane of the thigh of the user measured by an inertial measurement device attached to the thigh of the user.

Specifically, the angular velocity may be defined as an angular velocity for the sagittal plane of the thigh, which is the area assisted by the wearable robot 200 according to the present invention. The angular velocity may be measured to recognize the angular velocity of the thigh that moves back and forth of the body when the user wearing the wearable robot 200 walks. The computing device 100 or the wearable robot 200 may collect 400 thigh sagittal plane angular velocities per second through the inertial measurement device (an inertial measurement unit: IMU) attached to the center of the front part of the user's thigh.

In one embodiment, the delivered force may include a force exerted by the mobile wearable robot worn on the user to pull the thigh of the user correspondingly to the assistance power.

Specifically, the delivered force may be defined as a force delivered to the user wearing the wearable robot 200 to assist the lower limbs. For example, when a user wears the wearable robot 200 according to the present invention, an actuator (a motor) and the back of the user's thigh may be connected with a Bowden cable. In this case, the wearable robot 200 may assist the user's walking by rotating the actuator in accordance with the walking under the control of the computing device 100 to wind the Bowden cable, thereby pulling the back of the thigh. In this process, the force transmitted to the back of the thigh as the cable is pulled may be a delivered force.

In one embodiment, the force timing may include a time of providing the user with the assistance power based on a gait cycle percentage recognized from a difference between a first time at which a maximum angle is recognized by the inertial measurement device and a second time at which a next maximum angle is recognized.

Specifically, the force timing may be defined as the timing of transmitting the delivered force. The wearable robot 200 according to the present invention needs to identify a gait cycle percentage (GCP (%)) in real time to effectively assist lower limb walking assistance. To this end, the computing device 100 or the wearable robot 200 may calculate the gait cycle percentage by setting 0% at Maximum Hip Flexion (MHF), which is a state in which the thigh stretches highest forward during a stride and 100% at the next MHF timing, and may be measured repeatedly at each step of walking.

Specifically, the computing device 100 or the wearable robot 200 may calculate the gait cycle percentage in real time using an angle value for a sagittal plane measured by the inertial measurement device attached to the user's thigh. Additionally, the computing device 100 or the wearable robot 200 may provide assistance power to the user when the gait cycle percentage is reached at a preset force timing based on the real-time gait cycle percentage.

The computing device 100 may, upon collection of the body response information of the user, perform high-speed optimization on an auxiliary profile corresponding to assistance power based on the body response information (S130). Additionally, the computing device 100 may provide the user with optimal assistance power corresponding to an optimal auxiliary profile on which the high-speed optimization has been performed (S140).

In the present invention, the auxiliary profile may refer to a series of parameters or settings that define the characteristics of the assistance power provided by the wearable robot 200 to the user.

For example, an auxiliary profile may be composed of parameters including the point in time when power is transmitted to the user (the point in time when a delivered force is generated), the point in time when the magnitude of the delivered force is greatest, and the point in time when the delivered force becomes zero again. Additionally, the parameters that compose the auxiliary profile may include an force timing to determine the point in time when to provide the assistance power in the gait cycle percentage, the magnitude and intensity of the assistance power, the duration of the assistance power, and the form and pattern of providing the assistant power, such as linear assistance, gradually increase or decrease, momentary pulses, and the like.

The auxiliary profile may be dynamically adjusted while monitoring the user's state and the environment in real time through sensors and algorithms, and may be continuously optimized to provide the user with the most appropriate walking assistance.

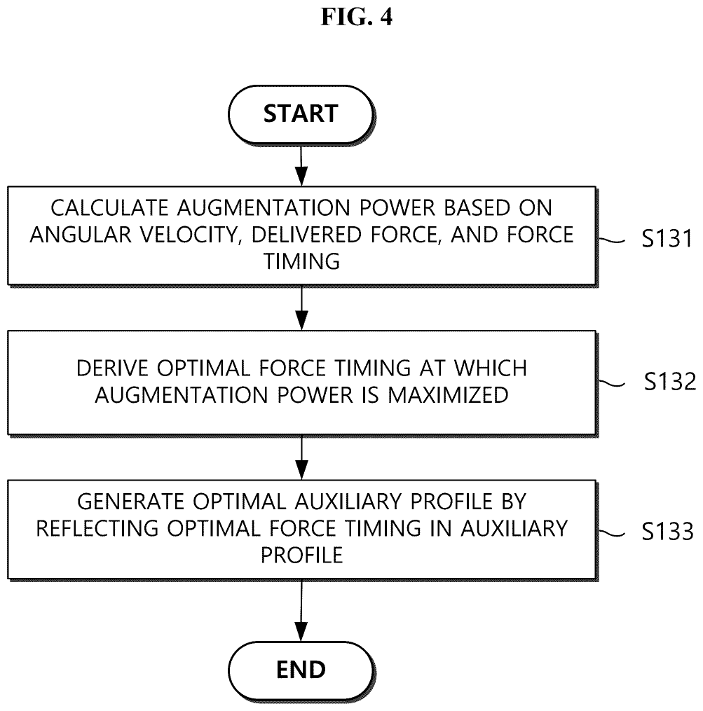

Referring to FIG. 4, the computing device 100 may, when performing high-speed optimization on the auxiliary profile corresponding to the assistance power based on the body response information in operation S130, calculate an augmentation power based on the angular velocity, the delivered force, and the force timing (S131).

Specifically, the computing device 100 may, when calculating the positive augmentation power based on the angular velocity, the delivered force, and the force timing, recognize the delivered force transmitted to the user correspondingly to the force timing during the gait cycle percentage of the user.

The computing device 100 may recognize a moment arm, which is the distance between the point of action of the user's body corresponding to the delivered force and the center of gravity of the user's thigh.

The computing device 100 may perform integration on a value obtained by multiplying the delivered force and the moment arm, and may recognize augmentation work generated during the gait cycle percentage by the delivered force.

The computing device 100 may calculate an augmentation power by dividing the augmentation work by a time corresponding to the gait cycle percentage.

That is, the computing device 100 may calculate the augmentation power obtained by normalizing the augmentation work generated during the gait cycle percentage with respect to the walking time.

The computing device 100 may, upon calculation of the augmentation power, derive an optimal force timing at which the augmentation power is maximized (S132).

Specifically, the computing device 100 may, when deriving the optimal force timing at which the augmentation power is maximized, obtain a dataset including a plurality of force timings previously provided to the user and augmentation power corresponding to the plurality of force timings, respectively.

Meanwhile, when there is no previous history of assistance power provided to the user according to a plurality of force timings, the computing device 100 may build a dataset by providing a user with assistance power according to randomly generated force timings a preset number of times and obtaining augmentation power.

The computing device 100 may repeatedly perform a Bayesian optimization technique based on the dataset to derive a force timing that maximizes an expected augmentation power value.

In one embodiment, the Bayesian optimization technique used by the computing device 100 may include three processes: a process of obtaining a surrogate model, a process of deriving an optimal point through an objective function (an acquisition function), and a process of updating the dataset with an optimal point and performing iteration.

In the process of obtaining a surrogate model according to the present invention, the surrogate model may be a function for identifying the trend of the force timing, which is a parameter, and the augmentation power, which is an objective function, using the dataset. For example, the computing device 100 may obtain a surrogate model using a Gaussian process regression (GPR) function. Additionally, the computing device 100 may, through the GPR function, achieve prediction even for an area in which the trend of an augmentation power according to a force timing is not provided as data.

In order to obtain a GPR function that accurately predicts the trend, the computing device 100 trains a configuration value of an internal Kernel function inside the GPR, which is responsible for the prediction performance of the GPR function, with an existing dataset to obtain the optimal value of the kernel function. Through this, the computing device 100 may obtain a surrogate model function that accurately predicts the relationship and trend between the force timing and the augmentation power based on the dataset.

In the process of deriving the optimal point according to the present invention, the objective function may be a function used to search for the force timing at which the maximum augmentation power is applicable in the GPR function. The computing device 100 according to the present invention may use an expected improvement (EI) function as the objective function. The computing device 100 may derive the optimal point (a force timing value at which the augmentation power is maximized) from the GPR function through the corresponding objective function. Here, the expected improvement function may be configured to derive the optimal point by adjusting the importance in an uncertain area (an area in which the augmentation power value needs to be predicted due to absence of data) and a certain area (an area in which the augmentation power value is known due to presence of data) of the GPR function using a parameter referred to as xi, to derive an appropriate optimal point according to each individual and each walking environment. Through this, the computing device 100 may, by utilizing the trained GPR function, derive a force timing value at which the maximum augmentation power is predicted to be applied.

In the iterative process according to the present invention, the computing device 100 may update the dataset with the optimal point derived through the previous process and repeat the above processes to search for a better optimal point. As more data (optimal points obtained in the previous process) are included in the dataset, the surrogate model may more accurately predict the relationship and trend between the force timing and the augmentation power, and the objective function may also derive a better optimal point through a more accurate surrogate model.

The computing device 100 may generate an optimal auxiliary profile by reflecting the optimal force timing in the auxiliary profile (S133).

Specifically, the computing device 100 may, when generating an optimal auxiliary profile by reflecting the optimal force timing in the auxiliary profile, adjust parameters of the auxiliary profile based on the optimal force timing. Here, the parameters of the auxiliary profile may include the point in time when the delivered force is generated, the point in time when the magnitude of the delivered force is greatest, and the point in time when the delivered force becomes 0 again.

For example, the computing device 100 may adjust the parameters of the auxiliary profile based on the optimal force timing, thereby more precisely adjusting the point in time when the assistance power is generated, the point in time when the maximum force is transmitted, and when the force transmission ends, etc., in synchronization with the gait cycle percentage of the user.

Through the adjustment, the computing device 100 and the wearable robot 200 may provide an auxiliary profile optimized for the user's walking pattern, to improve the user's walking efficiency and minimize energy consumption that may occur during walking, thereby maximizing the user's walking stability and comfort.

Therefore, the present invention may provide a personalized walking assistance solution, enabling users with various walking abilities to engage more actively in their daily lives.

According to various embodiments of the present invention, some of the operations described above may be performed repeatedly.

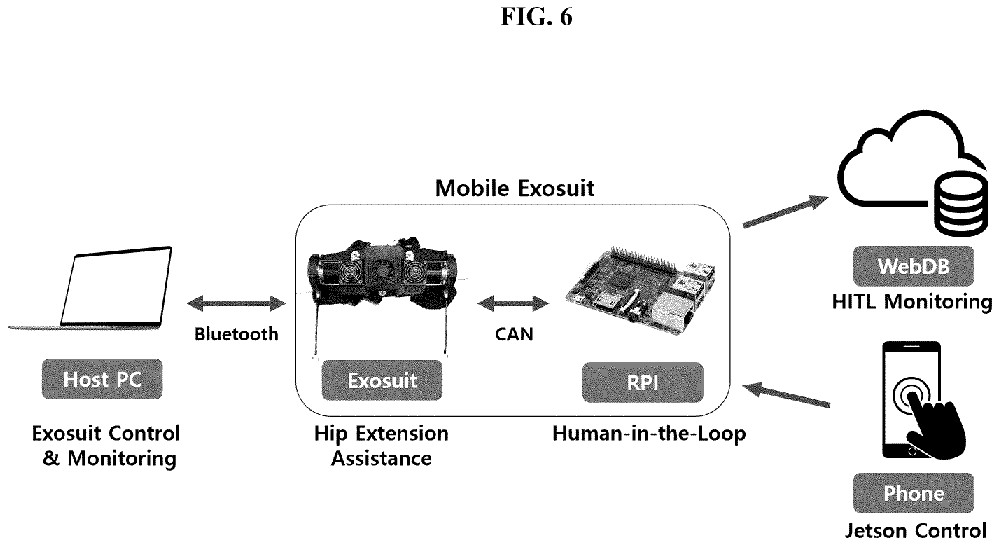

Referring to FIG. 6, the computing device 100 may, after providing the user with the optimal assistance power, perform high-speed optimization again using the user's body response information measured after providing the optimal assistance power (S210). Additionally, the computing device 100 may provide the user with assistance power corresponding to the optimal auxiliary profile on which the high-speed optimization has been performed (S220).

In other words, the computing device 100 may continuously collect and analyze data related to the user's walking, thereby dynamically adjusting an auxiliary profile optimized based on an in-depth understanding of a current walking state and a previous walking pattern of the user. This process may make it possible to update and improve the auxiliary profile in real time in response to various walking conditions and environmental changes that the user may experience.

As a result, the approach according to the present invention may provide users with more effective and comfortable walking assistance and contribute to improving the user's walking ability in the long term.

In additional embodiments, the present invention may improve the user experience of walking assistance technology by providing assistance tailored to the user's individual needs and preferences, thereby enhancing accessibility to a wider range of users.

For example, a patient undergoing rehabilitation treatment may require intensive training for specific muscle groups or walking stages. When a patient with hemiplegia due to a stroke uses the wearable robot 200 to recover the walking ability, the use may be not only to simply assist walking, but also to rehabilitate and strengthen the damaged nervous system and muscles. Accordingly, the computing device 100 according to the present invention may analyze the patient's walking pattern in real time to generate a customized auxiliary profile, such as by providing more assistance power to the weakened leg or increasing assistance power at a specific stage of the gait cycle percentage. In this way, the computing device 100 may optimize the auxiliary profile according to the patient's individual needs and rehabilitation goals, resulting in a more efficient rehabilitation process and faster recovery.

As another example, different daily activities, such as walking on a flat ground, climbing stairs, or walking on an irregular ground, may require different auxiliary profiles. Accordingly, the wearable robot 200 according to the present invention may detect the type of activity in which the user is currently engaged and automatically select an auxiliary profile appropriate for the type of activity. For example, the wearable robot 200 may provide more assistance power when the user climbs stairs and switch to a profile that induces a more natural walking pattern on a flat ground. This allows users to receive optimal walking assistance in a variety of environments, thereby expanding the range of activities in daily lives and improving the user's independence.

Through this, the present invention may optimize the auxiliary profile at high speed to provide assistance power that corresponds to the user's personal needs and preferences. This not only improves the user's walking efficiency, minimizes energy consumption during walking, and maximizes the user's walking stability and comfort, but also improves the efficiency of the rehabilitation process, or allows the user to be more active in daily lives.

In various embodiments, referring to FIG. 6, the present invention may implement a human-in-the-loop by linking the existing wearable robot and a Raspberry Pi and utilizing data from wearables. Here, the human-in-the loop is a form of computing system in which an artificial intelligence system and humans interact, and may include a system that assists the user's walking.

In one embodiment, the present invention may include a host PC communication system capable of remotely monitoring and controlling up to about 50 meters using Bluetooth.

Additionally, the present invention may be configured to control the Raspberry Pi with the user terminal 300. Additionally, the present invention may be configured such that real-time optimization status may be checked anytime, anywhere by linking the web and Raspberry Pi in real time.

Therefore, the present invention may provide a mobile human-in-the-loop robot system that may be customized to the wearer in real time, anytime anywhere.

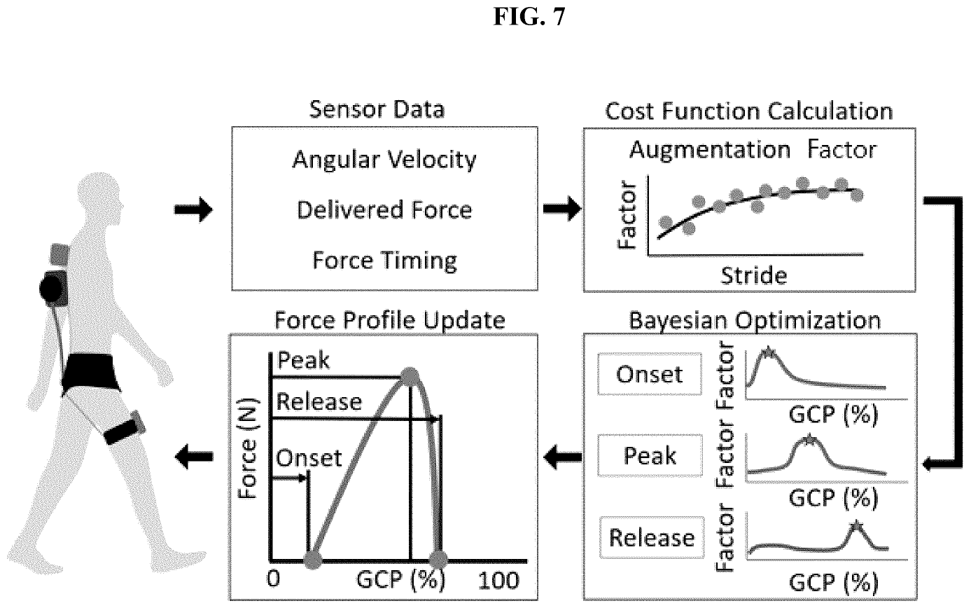

In various embodiments, referring to FIG. 7, the computing device 100 according to the present invention may use a Bayesian optimization algorithm as an algorithm for building a high-speed human-in-the-loop system.

Specifically, the computing device 100 may perform a Bayesian optimization algorithm based on a power-based Augmentation Factor representing the assistance performance of the wearable.

Therefore, the present invention may perform high-speed optimization in real time to search for and present the optimal profile at a high speed.

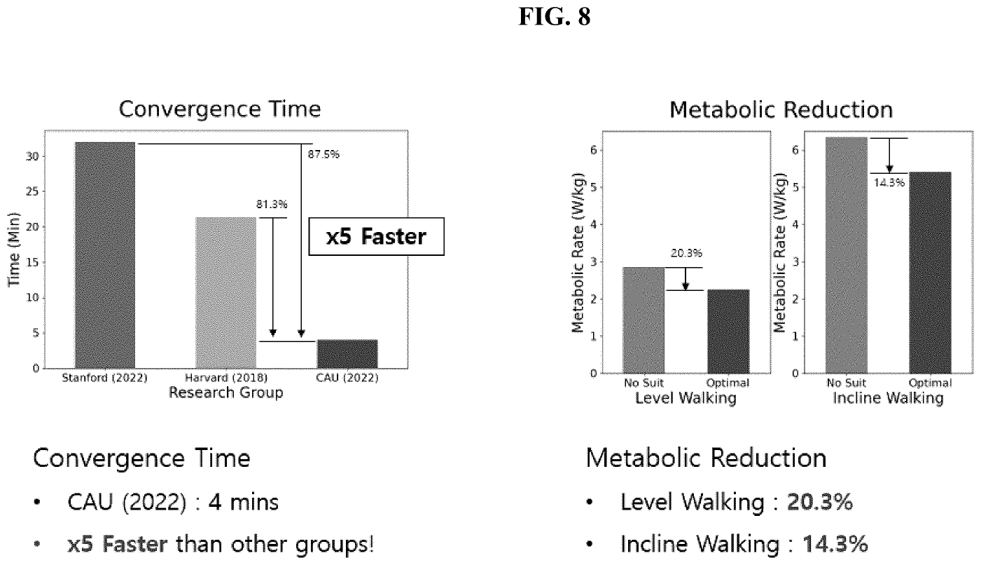

In various embodiments, referring to FIG. 8, the present invention (represented by CAU on the graph) achieved an optimization speed about 5 times faster than that of the existing research group, and as a result of the optimization, achieved reductions in metabolic energy of 20.3% and 14.3% for walking on a flat ground and walking on a uphill, respectively.

FIG. 9 is a diagram illustrating an example of a method of predicting a gait cycle percentage corresponding to various walking environments according to various embodiments according to the present invention.

Referring to FIG. 9, the computing device 100 may obtain sensing data according to the user's walking from the inertial measurement device attached to the user's thigh (S310).

In the present invention, the sensing data may include the angle and the angular velocity for the sagittal plane of the user's thigh.

Specifically, the computing device 100 or the wearable robot 200 may collect 400 angles and angular velocities on the sagittal plane of the thigh per second through the inertial measurement device (IMU) attached to the center of the front part of the user's thigh.

The computing device 100 may define a plurality of gait feature points based on the sensing data (S320).

Specifically, the computing device 100 may, when defining the plurality of gait feature points based on the sensing data, define the lowest point of the angle as a first gait feature point. For example, the first gait feature point may be defined as a maximum hip extension (MHE).

Additionally, the computing device 100 may, upon defining of the first gait feature point, define the highest point of the angular velocity as a second gait feature point. For example, the second gait feature point may be defined as a peak velocity of the angular velocity.

Additionally, the computing device 100 may, upon recognition of the second gait feature point, define the highest point of the angle as a third gait feature point. For example, the third gait feature point may be defined as a maximum hip flexion (MHF).

Additionally, the computing device 100 may define a valley point that appears immediately after the third gait feature point as a fourth gait feature point. For example, the fourth gait feature point may be defined as a minimum hip flexion (MinHF).

The computing device 100 may, upon defining of the plurality of gait feature points, determine a specific gait feature point corresponding to the current walking environment of the user based on the plurality of gait feature points and reference values preset for a plurality of walking environments, respectively (S330).

Specifically, the computing device 100 may, when determining a specific gait feature point, set a candidate region based on four reference values preset for a plurality of walking environments. Here, the four reference values may be changed based on the angle and the angular velocity included in the sensing data.

For example, the four reference values th1, th2, th3, and th4 may respond to changes in user's walking states and environments.

In more detail, for example, the computing device 100 may identify the MHE by searching for the lowest point of the thigh angle during walking through the first reference value th1, and adjust the first reference value th1 during walking of an uphill or when the walking speed changes. In addition, the computing device 100 may determine the Peak Velocity by identifying the highest point of the angular velocity through the second reference value th2, and may adjust the second reference value th2 according to the changing walking environment. In addition, the computing device 100 may identify the MHF by searching for the highest point of the thigh angle during walking through the third reference value th3, and may adjust the third reference value th3 to match various walking patterns of the user. Additionally, the computing device 100 may identify the MinHF by analyzing a change in angle through the fourth reference value th4, and may adjust the fourth reference value th4 by reflecting changes in the walking environment. Through the reference values, the computing device 100 may determine a specific gait feature point that match with the current walking environment of the user, but is not limited thereto.

In one embodiment, the four reference values described above may form a candidate region in the graph.

The computing device 100 may define the highest point of the angular velocity present within the candidate region as a fifth gait feature point and evaluate the fifth gait feature point. For example, the fifth gait feature point may be defined as a feature peak (FP) of the angular velocity.

Specifically, the computing device 100 may, when evaluating the fifth gait feature point, recognize whether the fifth gait feature point is present at a time preceding the third gait feature point (MHF).

The computing device 100 may recognize whether the fifth gait feature point is present outside the candidate region. For example, the computing device 100 may recognize whether the fifth gait feature point is larger than the candidate region and is thus present outside the candidate region, or whether the fifth feature point is present earlier or later than the candidate region.

The computing device 100 may recognize whether a plurality of fifth gait feature points are present in the candidate region.

The computing device 100 may recognize whether the fifth gait feature point is present a preset time later than the fourth gait feature point (MinHF).

The computing device 100 may, after completing the evaluation of the fifth gait feature point, determine a specific gait feature point corresponding to the current walking environment based on the result of evaluating the fifth gait feature point.

Specifically, the computing device 100 may, when determining a specific gait feature point corresponding to the current walking environment based on the result of evaluating the fifth gait feature point, determine a specific gait feature point as a point corresponding to the third gait feature point or the fifth gait feature point based on the result of evaluating the fifth gait feature point.

For example, the computing device 100 may, in response to the position of the specific gait feature point corresponding to the third gait feature point, recognize the current walking environment of the user as a uphill walking. Additionally, the computing device 100 may, in response to the position of the specific gait feature point corresponding to the fifth gait feature point present in the candidate region, recognize the current walking environment of the user as walking on a flat ground or running.

The computing device 100 may, upon determination of a specific gait feature point corresponding to the current walking environment, predict the gait cycle percentage of the user based on the specific gait feature point (S340).

In one embodiment, the computing device 100 may, when predicting the gait cycle percentage of the user based on the specific gait feature point, predict the time difference between a currently determined N^{th} specific gait feature point and a N−1^{th} specific gait feature point determined immediately before the N^{th} specific gait feature point as the gait cycle percentage of the user.

Through this method, the computing device 100 may analyze and understand the user's walking pattern in real time while increasing the accuracy of the gait cycle percentage. Furthermore, based on the analysis, the computing device 100 may provide users with more personalized walking support and improve the user's walking safety and efficiency through adjustment of the wearable robot 200. For example, when the wearable robot 200 recognizes that the user is walking an uphill, the wearable robot 200 may provide the user with additional support (e.g., stronger assistance power) to facilitate walking.

Additionally, the present invention may provide deep insight into the user's walking, thereby aiding in early identification and prevention of potential walking-related issues. For example, abnormal feature points consistently found in walking patterns may indicate potential health issues, enabling provision of important feedback to users or medical professionals.

In summary, an embodiment of the present invention provides an effective method of analyzing and predicting a user's walking with high precision. This may improve the user's walking experience, minimize walking-related risks, and have a positive impact on overall health and well-being.

According to various embodiments of the present invention, the computing device 100 may, upon prediction of the user's gait cycle percentage, correct an error to improve the accuracy of the gait cycle percentage.

The computing device 100 may, upon prediction of the user's gait cycle percentage, calculate a gait cycle percentage error for the gait cycle percentage based on the angle included in the sensing data. Additionally, the computing device 100 may correct the predicted gait cycle percentage by compensating for the error.

Specifically, the computing device 100 may, when calculating an error for the gait cycle percentage, input a signal corresponding to the angle to an oscillator to obtain a phase value corresponding to the angle.

In the present invention, the oscillator may include an adaptive oscillator (AO). The AO may have characteristics of defining any input signal as a combination of trigonometric functions and interpreting the frequency and phase values of a main signal. The computing device 100 according to the present invention may use the characteristics of the AO to interpret the frequency and phase values of the signal corresponding to the angle included in the sensor data.

For example, the AO may obtain a phase, an amplitude, a frequency, etc., of the angle signal based on the equation below.

$$\hat{θ} ( t ) = {α}_{1} ( t ) sin ( {φ}_{1} ( t ) ) + {α}_{2} ( t ) sin ( {φ}_{2} ( t ) ) + {α}_{3} ( t ) sin ( {φ}_{3} ( t ) ) + {α}_{0} ( t )$$

\tilde{θ}(t): A sin wave combination formula that simulates the angle data obtained through AO

$${\dot{φ}}_{i} ( t ) = ω ( t ) * i + {v}_{φ} \frac{F ( t )}{\sum {α}_{i}} cos ( {φ}_{i} ( t ) )$$

$$\dot{ω} ( t ) = {v}_{ω} \frac{F ( t )}{\sum {α}_{i}} cos ( {φ}_{1} ( t ) )$$

$${\dot{α}}_{i} ( t ) = η F ( t ) sin ( {φ}_{i} ( t ) )$$

$${\dot{α}}_{0} ( t ) = η F ( t )$$

$$F ( t ) = θ ( t ) - \tilde{θ} ( t )$$

φᵢ(t): A phase of an angle signal
αᵢ(t): An amplitude of an angle signal
ωᵢ(t): A frequency of an angle signal
F(t). A difference between magnitudes of an actual signal and an AO signal
   η, ν_{ω}, ν_{φ}: A gain value of each signal

In addition, the AO may, whenever new angle data is received, update an AO equation based on a preset gain value of each signal based on the above equation, allowing for accurate prediction of the main frequency and the phase values of the angle data.

The computing device 100 according to the present invention may calculate the gait cycle percentage error according to conditions based on the magnitude of the corresponding phase, using the following equation.

$${φ}_{n} ( t ) = mod ( {φ}_{1} ( t ) , 2 π )$$

$${P}_{e} ( {t}_{k} ) = \{ \begin{matrix}- {φ}_{n} ( {t}_{k} ) & if 0 \leq {φ}_{n} ( {t}_{k} ) < π \\ 2 π - {φ}_{n} ( {t}_{k} ) & if π \leq {φ}_{n} ( {t}_{k} ) < 2 π\end{matrix}$$

$$( {t}_{k} ) = \{ \begin{matrix}{P}_{e} ( {t}_{k} ) - 2 π & if {P}_{e} ( {t}_{k} ) > \frac{π}{2} and ( {t}_{k} - 1 ) < - \frac{π}{2} \\ {P}_{e} ( {t}_{k} ) + 2 π & if {P}_{e} ( {t}_{k} ) < - \frac{π}{2} and ( {t}_{k} - 1 ) > \frac{π}{2} \\ {P}_{e} ( {t}_{k} ) & otherwise\end{matrix}$$

tₖ: The time at which the k^{th} HS was acquired

Here, [symbol] (tₖ) represents the gait cycle percentage error.

Additionally, the computing device 100 may, upon calculation of a gait cycle percentage error based on the phase value, correct the predicted gait cycle percentage by compensating for the error.

Specifically, the computing device 100 may perform gait cycle percentage correction through errors using the following equation.

$$∅ ( t ) = {φ}_{n} ( t ) - ( {t}_{k} )$$

$${\dot{φ}}_{e} = {k}_{p} [ ( {t}_{k} ) - {φ}_{e} ( {t}_{k} ) ] * ω * {e}^{- ω ( t ) ( t - {t}_{k} )}$$

$$∅ ( t ) = mod ( {φ}_{n} ( t ) - {φ}_{e} ( t ) , 2 π )$$

ø(t): A phase prediction for angular data
φₑ: A gait cycle percentage error compensation value
   kₚ: A compensation gain value

In other words, the computing device 100 according to the present invention may first derive a gait cycle percentage based on a heel strike (i.e., a specific gait feature point) by utilizing an angle value and an angular velocity value through a gait cycle percentage prediction algorithm. Additionally, the computing device 100 may obtain the main frequency value and the main phase value of the signal by analyzing the signal with respect to the angle of the sagittal plane of the thigh through the AO. In addition, the computing device 100 may calculate the error value of the gait cycle percentage based on the obtained phase value, and compensate for the error of the gait cycle percentage in real time through a correction equation to predict the gait cycle percentage more accurately.

The above described method of predicting a gait cycle percentage corresponding to a walking environment may form a basis for the computing device 100 to understand and improve a user's walking pattern in more detail. In particular, analysis using an AO enables support deeply customized to each user's walking characteristics, which may help users maintain a more natural and efficient walking.

According to various embodiments of the present invention, walking assistance and analysis functions through interaction between the computing device 100 and the wearable robot 200 may be expanded. For example, by integrating machine learning algorithms and AI technology, analysis of walking data of users may be more precisely and dynamically performed. The technologies may identify subtle changes in a user's walking pattern, thereby providing more accurate prediction of walking environments and personalized walking support strategies.

In addition, since predictive maintenance of the user's walking state is possible by utilizing real-time data analysis and prediction models, potential problems that may occur during walking can be prevented. For example, analyzing walking patterns may predict the likelihood of future health issues and provide warnings to users or relevant medical professionals.

In addition, by developing the sensor technology of the wearable robot 200, a method of interacting with the user's environment may be upgraded. For example, the user's walking environment (e.g., a slope of the ground, slipperiness, etc.) may be more accurately recognized through an external environmental sensor, and accordingly the strength of walking assistance may be adjusted. Such a function may ensure users to walk safely in various environments.

Additionally, through a haptic feedback system integrated into the wearable robot 200, intuitive walking assistance may be provided to the user. The system may inform users of the optimal time or direction to move the feet during walking, thereby improving walking efficiency and reducing fatigue.

In various embodiments, the gait cycle percentage may be measured based on gait feature points that appear during walking and may be measured based on the heel strike HS, which is the moment when the foot steps on and touches the ground.

In other words, the gait cycle percentage may be obtained such that when the interval between the HS of the previous step and the HS of the current step is represented as 100%, the interval is repeatedly predicted to calculate the gait cycle percentage.

Therefore, accurately identifying the gait feature point (e.g., the HS) may be important in predicting an accurate gait cycle percentage.

In various embodiments, in order to accurately predict gait feature points, the present invention has calculated, through experiments, how the heel strike (i.e., a gait feature point) appears in various walking environments. The present invention has found that the heel strike appears at different gait feature points in uphill walking, flat ground, and running walking.

Specifically, when the user walked uphill, the heel strike appeared at the MHF point, and when the user walked or ran on a flat ground, the heel strike appeared at the Feature Peak point.

Accordingly, the present invention first identifies the current walking environment through an adaptive threshold technique, and performs computations to predict which feature point is a heel strike according to the identified walking environment.

In various embodiments, the present invention may identify the walking environment and then recognize and/or analyze signal characteristics of the IMU sensor used for the gait cycle percentage based on an AO algorithm. Additionally, based on the result, the present invention may predict the gait cycle percentage in real time and calculate the error for the gait cycle percentage.

In other words, the present invention may compensate for errors based on the characteristics of the AO to correct the existing gait cycle percentage, thereby predicting a more accurate gait cycle percentage in real time.

FIG. 10 is a diagram illustrating an example of a method of simultaneously optimizing the energy efficiency of a wearable robot and the metabolic energy efficiency of a wearer according to various embodiments according to the present invention.

Referring to FIG. 10, the computing device 100 may generate a first function corresponding to metabolic energy and a second function corresponding to robot energy (S410).

Specifically, the computing device 100 may, when generating a first function corresponding to metabolic energy and a second function corresponding to robot energy, obtain exercise data and characteristic data of the user.

For example, the computing device 100 may recognize the exercise data and the characteristic data of the user using the wearable robot 200, or may receive at least a portion of the exercise data and the characteristic data of the user from the user terminal 300.

In one embodiment, the exercise data of the user may include information related to activities or exercises performed by the user while wearing the wearable robot 200. For example, exercise data may include data regarding the type of an activity (e.g., the type of exercise, such as walking, running, climbing stairs, etc.), the intensity of exercise (e.g., a speed, a distance, a duration of activity, etc.), and motion pattern data. (e.g., a movement pattern, a stride length, a stride frequency, etc., of a user) and biomechanical data (e.g., an joint angle, a muscle strength, an impact force generated during walking, etc.), but is limited thereto.

In one embodiment, the characteristic data of the user may include information about the physical and physiological characteristics of the individual using the wearable robot 200. For example, the characteristic data may include body measurement data (e.g., physical characteristics, such as the height, the weight, the limb length, etc.), age and sex data, and health status data (e.g., existing health issues, a muscle mass, a body fat percentage, etc.) and fitness level data (e.g., a fitness level, such as an athletic ability, an endurance, a strength, etc.), but is not limited thereto.

The computing device 100 may, upon obtaining of the exercise data and the characteristic data, generate a first function and a second function based on a regression model using the obtained exercise data and the obtained characteristic data. Here, the regression model may be a model to which zero mean settings and a specific kernel are applied. For example, the regression model may include, but is not limited to, a GPR model.

In one embodiment, each of the first function and the second function generated by the computing device 100 may output predicted values for each of the metabolic energy and the robot energy using a specific kernel.

The computing device 100 may, upon generating of the first function and the second function, recognize a Pareto front that minimizes each of the metabolic energy and the robot energy based on the first function and the second function (S420).

Specifically, the computing device 100 may, upon recognizing of the Pareto front, obtain a first prediction value of the metabolic energy based on the first function. Additionally, the computing device 100 may obtain a second prediction value of the robot energy based on the second function. Additionally, the computing device 100 may recognize a Pareto front that simultaneously minimizes each of the first prediction value and the second prediction value based on the first prediction value and the second prediction value.

For example, the computing device 100 may perform optimization in a direction that increases the area of a yellow rectangle generated from the Pareto front value found from an expected hypervolume improvement (EHVI) reference point.

In the present invention, the Pareto front is a concept used in a multi-objective optimization problem, and may refer to a set of solutions representing an optimal trade-off between conflicting goals. For example, solutions on the Pareto front are also referred to as “non-dominated solutions,” and no solution may completely dominate the other solutions. In other words, solutions on the Pareto front may be solutions that may not be better for all goals at the same time.

In one embodiment, the Pareto front may include predicted values in which the metabolic energy and the robotic energy are each minimized, and control variables. Here, the control variables may include an onset point, a peak point, a release point, an offset X value, and an offset Y value.

The computing device 100 may obtain an optimal auxiliary profile by adding a smooth value to an auxiliary profile based on the Pareto front (S430). Then, the computing device 100 may provide the user with assistance power corresponding to the optimal auxiliary profile (S440).

For example, the computing device 100 may obtain an optimal auxiliary profile by adding a smooth value to an auxiliary profile consisting of only an onset point, a peak point, and a release point.

In detail, for example, the computing device 10 may obtain the optimal auxiliary profile by adding an offset X value and an offset Y value corresponding to a smooth value to the onset point, the peak point, and the release point corresponding to the auxiliary profile.

In various embodiments, the computing device 100 may, when obtaining an optimal auxiliary profile by adding a smooth value to an auxiliary profile based on the Pareto front, recognize an onset point, a peak point, and a release point in the auxiliary profile. Additionally, the computing device 100 may add at least one of an offset X value and an offset Y value corresponding to the Pareto front to each of the onset point and the release point, thereby obtaining an optimal auxiliary profile.

According to various embodiments of the present invention, the computing device 100 may provide various modes of assistance power using various solutions included in the Pareto front.

Specifically, the computing device 100 may generate one or more optimal auxiliary profiles corresponding to a preset condition based on the Pareto front. Here, the one or more optimal auxiliary profiles may include an optimal auxiliary profile corresponding to at least one of a power mode, a smart mode, and an eco-mode.

For example, the computing device 100 may generate an optimal auxiliary profile corresponding to a power mode, a smart mode, and an eco-mode according to the weights of consideration given to the metabolic energy and the robotic energy among the Pareto fronts.

The computing device 100 may, when providing assistance power corresponding to the optimal auxiliary profile to the user, determine at least one mode based on the exercise data and the characteristic data of the user. Additionally, the computing device 100 may provide assistance power to the user based on the optimal auxiliary profile corresponding to the at least one mode.

For example, the computing device 100 may automatically select the most appropriate mode in consideration of the current activity state of the user, the physical characteristics of the user, surrounding environmental conditions, and the like, and provide the user with assistance power based on the optimal auxiliary profile matching with the corresponding mode.

In detail, for example, the computing device 100 may analyze the physical and physiological data of the user and the current activity state of the user in real time and automatically select the most appropriate mode (e.g., a power mode, a smart mode, and an eco-mode) at that moment. In this process, the computing device 100 may utilize a GPR model to search for the optimal balance between the metabolic energy of the user and the energy use efficiency of the wearable robot. For example, the computing device 100 may, when a user engages in high-intensity exercise, select the power mode to provide stronger assistance power, and for everyday activities, select the eco-mode to reduce energy consumption. Such a mode selection may be achieved by analyzing a variety of input variables, including exercise data (a speed, a distance, a duration, etc.) of the user, biomechanical data (a joint angle, a muscle strength, etc.) of the user, and body measurement data (a height, a weight, etc.) of the user. The computing device 100 may, through the analysis based on the data, derive and apply an auxiliary profile optimized for the user, thereby simultaneously maximizing the use effect and the energy efficiency of the wearable robot 200.

In one embodiment, the computing device 100 may perform deep learning-based time series analysis to select a mode appropriate for the user's current situation.

Specifically, the computing device 100 may process various input variables, such as the exercise data of the user, the biomechanical data of the user, and the body measurement data of the user, as time series data, and utilize a recurrent neural network (RNN) or a long short-term memory (LSTM) network. The networks are specialized in learning changes in data over time, allowing precise analysis of the current state and the activity pattern of the user. A pattern derived through the analysis may be used to determine the most appropriate mode for the user in real time. For example, the computing device 100 may automatically make a decision to select a power mode when the user's activity intensity increases and select an eco-mode when the user's activity intensity is low using an LSTM network.

Through this, the computing device 100 may provide energy-efficient and effective support according to various needs and situations of the user using the wearable robot 200, and simultaneously maximize user satisfaction and the use effect of the wearable robot 200.

In various embodiments, the computing device 100 according to the present invention may perform multi-objective Bayesian optimization to search for an optimal solution for correction of the auxiliary profile.

Specifically, EHVI may refer to an objective function used to optimize a single objective function used in a multi-objective optimization problem.

More specifically, the computing device 100 according to the present invention may derive a Pareto front, which is a set of solutions that may minimize two elements in response to various force profiles, with two objective functions (i.e., a first objective function corresponding to the energy efficiency of the wearable robot and a second objective function corresponding to the metabolic energy efficiency of the wearer) for which optimization is desired.

In various embodiments, the existing auxiliary profile designates points for each of Onset, Peak, and Release and utilizes a sine-cosine graph passing through the corresponding points. In this case, there may be a section in which the motor of the wearable robot 200 abruptly moves, requiring a significant amount of energy when the motor operates.

Accordingly, the present invention predicts that when a smooth value is added to the existing auxiliary profile to generate a graph that falls smoothly, energy may be saved even when performing the same exercise. Additionally, the present invention predicts that there are various methods of searching for the optimal solution for correction of the auxiliary profile.

In various embodiments, the computing device 100 according to the present invention may add at least one of an offset X and an offset Y to each of the onset point, the peak point, and the release point. Here, the offset X and the offset Y may each be determined based on a preset optimization algorithm. For example, the computing device 100 may set the onset, the peak, the release, and the offset X, and the offset Y as control values, and perform multi-objective Bayesian optimization to calculate the values of the offset X and the Offset Y, but it is not limited thereto.

Specifically, the computing device 100 according to the present invention may smoothly interpolate between data points using Cubic Hermite interpolation.

In various embodiments, the computing device 100 may configure auxiliary profiles in three modes. The present invention may generate auxiliary profiles in a power mode that maximizes the wearer's metabolic energy efficiency, an eco-mode that maximizes the wearable robot's energy efficiency, and a smart mode that maximizes the wearable robot's energy efficiency and the wearer's metabolic energy efficiency.

For example, the power mode maximizes the power consumption of the robot but minimizes metabolic energy, providing users with a high assistance effect. The power mode may be suitable for use when the wearer's physical strength is very low or during intense exercise (e.g., walking on an incline, hiking, running, etc.).

Additionally, the eco-mode may be a mode that maximizes the operating time of the robot by minimizing the power consumption of the robot. The eco-mode may be suitable for situations in which the system battery is low or the wearer's physical strength is high.

Meanwhile, the smart mode is an intermediate state between the power mode and the eco-mode, and may be a balanced mode considering both the system energy and the metabolic energy.

Such various mode switching may be actively changed by the computing device 100 or may be manually changed by the user.

As is apparent from the above, the present invention can optimize a robot according to human movement in real time through a method, an apparatus, and a program for high-speed optimization of an auxiliary profile of a mobile wearable robot.

The effects of the present disclosure are not limited to the effects described above, and other effects that are not described will be clearly understood by those skilled in the art from the above detailed description.

Although exemplary embodiments of the present invention have been described in detail above with reference to the accompanying drawings, those of ordinary skill in the technical field to which the present invention pertains should be able to understand that various modifications and alterations may be made without departing from the technical spirit or essential features of the present invention. Therefore, it should be understood that the embodiments described above are illustrative in all respects rather than restrictive.

What is claimed is:

1. A method of high-speech optimization of an auxiliary profile of a mobile wearable robot, which is performed by a computing device including at least one processor, the method comprising:
providing assistance power to a user to help the user's walk;
collecting body response information of the user measured after the providing of the assistance power;
performing high-speed optimization on an auxiliary profile corresponding to the assistance power based on the body response information; and
providing the user with optimal assistance power corresponding to the optimal auxiliary profile on which the high-speed optimization has been performed.

2. The method of claim 1, wherein the collecting of the body response information of the user in real time includes collecting the body response information including an angular velocity measured in a sagittal plane of a thigh of the user, a delivered force corresponding to the assistance power, and an force timing related to a time for delivering the assistance power to the user.

3. The method of claim 2, wherein the angular velocity includes an angular velocity for the sagittal plane of the thigh of the user measured by an inertial measurement device attached to the thigh of the user,
the delivered force includes a force exerted by a mobile wearable robot worn on the user to pull the thigh of the user in response to the assistance power, and
the force timing includes a time of providing the assistance power to the user based on a gait cycle percentage recognized from a difference between a first time at which a maximum angle is recognized by the inertial measurement device and a second time at which a next maximum angle is recognized.

4. The method of claim 2, wherein the performing of the high-speed optimization on the auxiliary profile corresponding to the assistance power based on the body response information includes:
calculating augmentation power based on the angular velocity, the delivered force, and the force timing;
deriving an optimal force timing at which the augmentation power is maximized; and
generating the optimal auxiliary profile by reflecting the optimal force timing in the auxiliary profile.

5. The method of claim 4, wherein the calculating of the augmentation power based on the angular velocity, the delivered force, and the force timing includes:
recognizing a delivered force transmitted to the user correspondingly to the force timing during the gait cycle percentage of the user;
recognizing a moment arm, which is a distance between a point of action of a body of the user corresponding to the delivered force and a center of gravity of the thigh of the user;
performing integration on a value obtained by multiplying the delivered force and the moment arm and recognizing augmentation work generated during the gait cycle percentage by the delivered force; and
calculating the augmentation power by dividing the augmentation work by a time corresponding to the gait cycle percentage.

6. The method of claim 4, wherein the deriving of the optimal force timing at which the augmentation power is maximized includes:
obtaining a dataset including a plurality of force timings previously provided to the user and augmentation power corresponding to the plurality of force timings, respectively; and
repeatedly performing a Bayesian optimization technique based on the dataset to derive a force timing that maximizes an expected augmentation power value.

7. The method of claim 4, wherein the generating of the optimal auxiliary profile by reflecting the optimal force timing in the auxiliary profile includes adjusting parameters of the auxiliary profile based on the optimal force timing, and
the parameters of the auxiliary profile include a point in time at which a delivered force is generated, a point in time at which a magnitude of the delivered force is greatest, and a point in time at which the delivered force becomes 0 again.

8. The method of claim 1, further comprising:
after the providing of the optimal assistance power to the user, performing high-speed optimization again using body response information of the user measured after the providing of the optimal assistance power; and
providing the user with assistance power corresponding to the optimal assisting profile on which the high-speed optimization has been performed,
wherein the optimal auxiliary profile is optimized in real time and updated.

9. The method of claim 1, further comprising:
obtaining sensing data according to walking of the user from an inertial measurement device attached to the thigh of the user;
defining a plurality of gait feature points based on the sensing data;
determining a specific gait feature point corresponding to a current walking environment of the user based on the plurality of gait feature points and reference values preset for a plurality of walking environments, respectively; and
predicting a gait cycle percentage of the user based on the specific gait feature point.

10. The method of claim 1, further comprising:
generating a first function corresponding to a metabolic energy and a second function corresponding to a robotic energy;
recognizing a Pareto front that minimizes each of the metabolic energy and the robot energy based on the first function and the second function;
adding a smoothing value to an auxiliary profile based on the Pareto front to obtain an optimal auxiliary profile; and
providing the user with assistance power corresponding to the optimal auxiliary profile.

11. An apparatus comprising:
a memory in which one or more instructions are stored; and
a processor configured to execute the one or more instructions stored in the memory,
wherein the processor performs the method according to claim 1 by executing the one or more instructions.

12. A computer-readable recording medium on which a program for executing a method of providing a surgery simulation in conjunction with a computing device is recorded, wherein the method comprises:
providing assistance power to a user to help the user's walk;
collecting body response information of the user measured after the providing of the assistance power,
performing high-speed optimization on an auxiliary profile corresponding to the assistance power based on the body response information; and
providing the user with optimal assistance power corresponding to the optimal auxiliary profile on which the high-speed optimization has been performed.
